SEQUENCE LISTING (26 sequences)

SEQ ID NO: 1 - DNA, 24 bp, synthetic construct
  misc_feature 1..24: note = Single strand DNA oligonucleotide
  source 1..24: mol_type = other DNA
ataaccggtctccatcattgcagg

SEQ ID NO: 2 - DNA, 37 bp, synthetic construct
  misc_feature 1..37: note = Single strand DNA oligonucleotide
  source 1..37: mol_type = other DNA
atattgactttacccttaacaaagccatctataaaca

SEQ ID NO: 3 - DNA, 37 bp, synthetic construct
  misc_feature 1..37: note = Single strand DNA oligonucleotide
  source 1..37: mol_type = other DNA
ctttttagaagtttgtttatagatggctcttgttaag

SEQ ID NO: 4 - DNA, 75 bp, synthetic construct
  misc_feature 1..75: note = Single strand DNA oligonucleotide
  source 1..75: mol_type = other DNA
atattaattaatgacaagaacacgaactgagatggagtagtgatactgtaagatctatttaatacgaatctgaat

SEQ ID NO: 5 - DNA, 19 bp, synthetic construct
  misc_feature 1..19: note = Single strand DNA oligonucleotide
  source 1..19: mol_type = other DNA
ccctccaggttcgtttgtt

SEQ ID NO: 6 - DNA, 24 bp, synthetic construct
  misc_feature 1..24: note = Single strand DNA oligonucleotide
  source 1..24: mol_type = other DNA
tttgtttggtttcggcctattatt

SEQ ID NO: 7 - DNA, 28 bp, synthetic construct
  misc_feature 1..28: note = Single strand DNA oligonucleotide
  source 1..28: mol_type = other DNA
tgaagacataatggctgaaattcttctt

SEQ ID NO: 8 - DNA, 32 bp, synthetic construct
  misc_feature 1..32: note = Single strand DNA oligonucleotide
  source 1..32: mol_type = other DNA
tgaagacataagctcatttactcagcttttta

SEQ ID NO: 9 - DNA, 26 bp, synthetic construct
  misc_feature 1..26: note = Single strand DNA oligonucleotide
  source 1..26: mol_type = other DNA
tgaagacatacgatagacattgatcg

SEQ ID NO: 10 - DNA, 29 bp, synthetic construct
  misc_feature 1..29: note = Single strand DNA oligonucleotide
  source 1..29: mol_type = other DNA
tgaagacattcgtctctagacgtgtgagc

SEQ ID NO: 11 - DNA, 31 bp, synthetic construct
  misc_feature 1..31: note = Single strand DNA oligonucleotide
  source 1..31: mol_type = other DNA
tgaagacatcatataatggaagaatttcaag

SEQ ID NO: 12 - DNA, 27 bp, synthetic construct
  misc_feature 1..27: note = Single strand DNA oligonucleotide
  source 1..27: mol_type = other DNA
tgaagacattatgtccttttggcaagt

SEQ ID NO: 13 - DNA, 29 bp, synthetic construct
  misc_feature 1..29: note = Single strand DNA oligonucleotide
  source 1..29: mol_type = other DNA
gaaaactgccaaataatattgtcaagctc

SEQ ID NO: 14 - DNA, 29 bp, synthetic construct
  misc_feature 1..29: note = Single strand DNA oligonucleotide
  source 1..29: mol_type = other DNA
gagcttgacaatattatttggcagttttc

SEQ ID NO: 15 - DNA, 29 bp, synthetic construct
  misc_feature 1..29: note = Single strand DNA oligonucleotide
  source 1..29: mol_type = other DNA
catgattgagttctcccgttcaaatccta

SEQ ID NO: 16 - DNA, 29 bp, synthetic construct
  misc_feature 1..29: note = Single strand DNA oligonucleotide
  source 1..29: mol_type = other DNA
taggatttgaacgggagaactcaatcatg

SEQ ID NO: 17 - DNA, 30 bp, synthetic construct
  misc_feature 1..30: note = Single strand DNA oligonucleotide
  source 1..30: mol_type = other DNA
cagttgcttttctatgagctcattttaccc

SEQ ID NO: 18 - DNA, 30 bp, synthetic construct
  misc_feature 1..30: note = Single strand DNA oligonucleotide
  source 1..30: mol_type = other DNA
gggtaaaatgagctcatagaaaagcaactg

SEQ ID NO: 19 - DNA, 21 bp, synthetic construct
  misc_feature 1..21: note = Single strand DNA oligonucleotide
  source 1..21: mol_type = other DNA
atggtaccagagaaagagtgg

SEQ ID NO: 20 - DNA, 19 bp, synthetic construct
  misc_feature 1..19: note = Single strand DNA oligonucleotide
  source 1..19: mol_type = other DNA
ttcccggggatccgtcgac

SEQ ID NO: 21 - DNA, 30 bp, synthetic construct
  misc_feature 1..30: note = Single strand DNA oligonucleotide
  source 1..30: mol_type = other DNA
cttaacaagagccatcaagaaataaacttc

SEQ ID NO: 22 - DNA, 30 bp, synthetic construct
  misc_feature 1..30: note = Single strand DNA oligonucleotide
  source 1..30: mol_type = other DNA
gaagtttatttcttgatggctcttgttaag

SEQ ID NO: 23 - DNA, 30 bp, synthetic construct
  misc_feature 1..30: note = Single strand DNA oligonucleotide
  source 1..30: mol_type = other DNA
attgagtaagacatatttaatacgaatctg

SEQ ID NO: 24 - DNA, 30 bp, synthetic construct
  misc_feature 1..30: note = Single strand DNA oligonucleotide
  source 1..30: mol_type = other DNA
cagattcgtattaaatatgtcttactcaat

SEQ ID NO: 25 - protein, 861 aa, synthetic construct
  REGION 1..861: note = An exemplary amino acid sequence of wild-type Tm-2^2
MAEILLTSVINKSVEIAGNLLIQEGKRLYWLKEDIDWLQREMRHIRSYVDNAKAKEAGGDSRVKNLLKDIQELAGDVEDLLDDFLPKIQQSNKFNYCLKRSSFADEFAMEIEKIKRRVVDIDRIRKTYNIIDTDNNNDDCVLLDRRRLFLHADETEIIGLDDDFNMLQAKLLNQDLHYGVVSIVGMPGLGKTTLAKKLYRLIRDQFECSGLVYVSQQPRASEILLDIAKQIGLTEQKMKENLEDNLRSLLKIKRYVILLDDIWDVEIWDDLKLVLPECDSKVGSRMIITSRNSNVGRYIGGESSLHALQPLESEKSFELFTKKIFNFDDNNSWANASPDLVNIGRNIVGRCGGIPLAIVVTAGMLRARERTEHAWNRVLESMGHKVQDGCAKVLALSYNDLPIASRPCFLYFGLYPEDHEIRAFDLINMWIAEKFIVVNSGNRREAEDLAEDVLNDLVSRNLIQLAKRTYNGRISSCRIHDLLHSLCVDLAKESNFFHTAHDAFGDPGNVARLRRITFYSDNVMIEFFRSNPKLEKLRVLFCFAKDPSIFSHMAYFDFKLLHTLVVVMSQSFQAYVTIPSKFGNMTCLRYLRLEGNICGKLPNSIVKLTRLETIDIDRRSLIQPPSGVWESKHLRHLCYRDYGQACNSCFSISSFYPNIYSLHPNNLQTLMWIPDKFFEPRLLHRLINLRKLGILGVSNSTVKMLSIFSPVLKALEVLKLSFSSDPSEQIKLSSYPHIAKLHLNVNRTMALNSQSFPPNLIKLTLAYFSVDRYILAVLKTFPKLRKLKMFICKYNEEKMDLSGEANGYSFPQLEVLHIHSPNGLSEVTCTDDVSMPKLKKLLLTGFHCRISLSERLKKLSK

SEQ ID NO: 26 - DNA, 2586 bp, synthetic construct
  misc_feature 1..2586: note = An exemplary nucleic acid sequence which encodes wild-type Tm-2^2
  source 1..2586: mol_type = other DNA
atggctgaaattcttcttacatcagtaatcaataaatctgtagaaatagctggaaatttactgattcaagaaggaaagcgtttatattggttgaaagaggatatcgattggctccagagagaaatgagacacattcgatcttatgttgacaacgcaaaggccaaggaagctggaggtgattcaagggtcaaaaacttattgaaagatattcaagaattggcaggtgatgtggaggatctcttagatgacttccttccaaaaattcaacaatccaataagttcaattattgccttaagaggagttcttttgcagatgagtttgctatggagattgagaagataaagagaagggttgttgacattgaccgaataaggaaaacttacaacatcatagatacagataacaataatgatgattgtgttctgctggatcggagaagattattcctacatgctgatgaaacagagatcatcggtttggatgatgacttcaatatgctacaagccaaattacttaatcaagatttgcattatggagttgtttccatagttggcatgcccggtctggggaaaacaactcttgccaagaaactttataggctcattcgtgatcaatttgagtgttctggactggtctacgtttcacaacagccaagagcgagtgaaatcttacttgacattgccaaacaaattggactgacggaacagaaaatgaaggaaaatttggaggacaacctgcgatcactcttgaaaataaaaaggtatgttatcctcctagatgacatttgggatgtggaaatttgggatgatctgaaacttgtccttcctgaatgtgattcaaaagtcggcagtagaatgataatcacgtctcgaaatagtaatgtaggcagatacataggaggggaatcctccctccatgcattgcaacccctagaatccgagaaaagctttgaactctttaccaagaaaatctttaattttgatgataataatagttgggccaatgcttcacctgacttggtgaatattggtagaaatatagttgggagatgtggaggtataccgctagccatagtggtgactgcaggcatgttaagggcaagagaaagaacagaacatgcgtggaacagagtacttgagagtatgggccataaagttcaagatggatgtgctaaggtattggctctcagttacaatgatttacctattgcctcaaggccatgtttcttgtactttggcctttaccccgaggaccatgaaattcgtgcttttgatttgataaatatgtggattgctgagaagtttatagtagtaaatagtggtaataggcgagaggctgaggatttggcggaggacgtcctaaatgatttggtttctagaaacttgattcaacttgccaaaaggacatataatggaagaatttcaagttgtcgcatacatgacttgttacatagtttgtgtgtggacttggctaaggaaagtaacttctttcacaccgcgcatgatgcatttggtgatcccggcaatgttgctaggctccgaaggattacattctactctgacaatgtcatgattgagttcttccgttcaaatcctaagcttgagaagcttcgtgtacttttctgtttcgcaaaagacccttccatattttctcatatggcttattttgacttcaaattgttgcacacattggttgtagtcatgtctcaaagttttcaagcatatgtcactatcccaagcaaatttgggaacatgacttgcttacgctatctgagattggaggggaatatttgtggaaaactgccaaatagtattgtcaagctcacacgtctagagaccatagacattgatcgacgtagcctcattcaacctccttctggtgtttgggagtctaaacatttgagacatctttgttatagagattatggacaagcatgtaacagttgcttttctataagctcattttacccaaatatttactcattgcatcctaacaatctacaaaccttgatgtggatacctgataaattttttgaaccgaggttgttgcaccgattgatcaatttaagaaaactgggtatactgggagtgtccaattctaccgttaagatgttatcaatatttagccctgtgcttaaggcgctggaggttctgaagctcagtttttccagtgacccgagtgaacaaataaagttgtcatcgtatccacatattgctaagttgcatttgaatgttaacagaacaatggccttgaactctcaatcatttcctccaaatctcatcaagcttactctagcctactttagtgtagaccgttatatactggcagtacttaagacatttcccaaattaagaaaacttaaaatgttcatctgcaagtataatgaagaaaagatggatctctcgggcgaggcaaatggttatagctttccgcaacttgaagttttgcatattcatagcccgaatgggttgtctgaagtaacgtgcacggatgatgtcagtatgcccaaattgaaaaagctgttacttacaggattccattgccgaatcagtttatcggaacggcttaaaaagctgagtaaatga

(19) United States
(12) Patent Application Publication
(10) Pub. No.: US 2023/0329170 A1
(43) Pub. Date: Oct. 19, 2023

(54) TOBAMOVIRUS-RESISTANT TOMATO PLANTS

(71) Applicant: The State of Israel, Ministry of Agriculture & Rural Development, Agricultural Research, Rishon-LeZion (IL)

(72) Inventors: Ziv SPIEGELMAN, Rishon LeZion (IL); Hagit HAK, Gedera (IL); Ezra Joseph LOEB, North Bethesda, MD (US); Yifat SHERMAN, Modiin-Maccabim-Reut (IL)

(73) Assignee: The State of Israel, Ministry of Agriculture & Rural Development, Agricultural Research Organization, Rishon-LeZion (IL)

(21) Appl. No.: 18/142,065

(22) Filed: May 2, 2023

Related U.S. Application Data
(63) Continuation of application No. PCT/IL2021/051298, filed on Nov. 2, 2021.
(60) Provisional application No. 63/108,476, filed on Nov. 2, 2020.

(51) Int. Cl. A01H 1/00; A01H 6/82

(52) U.S. Cl. CPC A01H 1/126; A01H 6/825

(57) ABSTRACT

A tomato plant or a part thereof is disclosed. The plant expresses a Tm-2² protein having an amino acid sequence which renders the plant resistant to tomato brown rugose fruit virus (ToBRFV). Methods of generating same are also disclosed. Products generated therefrom are described.

### RELATED APPLICATIONS

This application is a Continuation of PCT Patent Application No. PCT/IL2021/051298 having International filing date of Nov. 2, 2021, which claims the benefit of priority under 35 USC § 119(e) of U.S. Provisional Patent Application No. 63/108,476 filed on Nov. 2, 2020. The contents of the above applications are all incorporated by reference as if fully set forth herein in their entirety.

### SEQUENCE LISTING STATEMENT

The XML file, entitled 96455SequenceListing.xml, created on Apr. 29, 2023, comprising 37,816 bytes, submitted concurrently with the filing of this application is incorporated herein by reference.

### FIELD AND BACKGROUND OF THE INVENTION

The present invention, in some embodiments thereof, relates to tomato plants which are resistant to the deleterious effects of tomato brown rugose fruit virus (ToBRFV).

Some of the more devastating plant viruses are members of the Tobamovirus genus, which includes tomato mosaic virus (ToMV), tobacco mosaic virus (TMV) and cucumber green mottle mosaic virus (CGMMV). Resistance against Tobamoviruses in tomato is conferred by the resistance (R) genes Tm-1, Tm-2 and Tm-2², all originating from wild tomato. In recent decades, resistance-breaking viral strains have emerged for Tm-1 and Tm-2, leaving Tm-2² as the key gene for control of Tobamovirus in tomato. Tm-2² encodes a member of the nucleotide-binding Leucine-rich repeat (NB-LRR or NLR) family of plant immune receptors. Members of the NLR family recognize specific pathogen-encoded effectors, termed avirulence (AVR) factors, either by directly binding to them or indirectly, via a mediator protein. This recognition triggers an immune signaling cascade that confines the pathogen to the infection site, typically through induction of programmed cell death (PCD) in a process termed hypersensitive response (HR).

Like many plant NLRs, Tm-2² contains a coiled-coil (CC) at its N-terminus and a centrally located NB domain, and an LRR domain at the C-terminus, which determines effector recognition specificity. To activate the antiviral immune response, Tm-2² associates with its AVR, the viral movement protein (MP). MPs are viral-encoded proteins that enables cell-to-cell transport of the virus via plasmodesmata, intercellular channels that connect between adjacent cells. Upon viral infection, Tm-2² directly binds to the Tobamovirus MP. This binding triggers the self-association of Tm-2² protein that allows the immune signal to occur.

Tm-2² is allelic to the broken resistance gene Tm-2. Interestingly, both alleles have different pathogen recognition capabilities. For example, Tm-2 confers resistance to the ToMV strain N3 but not to the strain B7, whereas Tm-2² protects against B7 but not against N3. Tm-2 and Tm-2² only differ in four amino acids, of which two are located in the NB domain and two are in the LRR domain. One of these LRR domain residues, Tyr-767, is essential for recognition of MP encoded by the ToMV strain B7, suggesting that specific residues within the Tm-2² LRR domain determine its ability to recognize specific MPs.

A recent outbreak of a new Tobamovirus named tomato brown rugose fruit virus (ToBRFV) has substantially damaged the tomato industry in Israel and Jordan. ToBRFV overcomes all known Tobamovirus resistances in tomato, including Tm-1, Tm-2 and Tm-2². Recent reports of ToBRFV outbreaks in France, Italy, Germany³, Holland, China, Mexico, Turkey and the USA indicate an emerging global epidemic. Sequence analysis showed that ToBRFV has 9-15% variance from other tobamoviruses such as TMV and ToMV, including 21 potential resistance-breaking mutations, 12 of them are located in the ToBRFV MP (MP^{ToBRFV}).

Background art includes Weber et al. (1993), Journal of virology, 67(11), 6432-6438 and Kobayashi, M., et al (2011), Journal of plant physiology, 168(10), 1142-1145.

### SUMMARY OF THE INVENTION

According to an aspect of the present invention, there is provided a tomato plant or a part thereof, expressing a Tm-2² protein having an amino acid sequence which renders the plant resistant to tomato brown rugose fruit virus (ToBRFV).

According to an aspect of the present invention, there is provided a cutting of a tomato plant described herein.

According to an aspect of the present invention, there is provided a seed of the plant described herein.

According to an aspect of the present invention, there is provided a cell having a genome of the plant described herein.

According to an aspect of the present invention, there is provided a culture comprising a plurality of cells described herein.

According to an aspect of the present invention, there is provided a method of breeding a tomato plant, comprising crossing the plant described herein with an additional tomato plant, thereby breeding the tomato plant.

According to an aspect of the present invention, there is provided a hybrid seed produced by the method described herein.

According to an aspect of the present invention, there is provided a hybrid plant, or part thereof, produced by growing the hybrid seed described herein.

According to an aspect of the present invention, there is provided a method of growing a plant, comprising vegetatively propagating the plant described herein, thereby growing the plant.

According to an aspect of the present invention, there is provided a food of processed product comprising the plant described herein or parts thereof.

According to embodiments of the present invention, the tomato plant is homozygotic for a Tm-2² mutation, which renders the plant resistant to tomato brown rugose fruit virus (ToBRFV).

According to embodiments of the present invention, the tomato plant is heterozygotic for a Tm-2² mutation, which renders the plant resistant to tomato brown rugose fruit virus (ToBRFV).

According to embodiments of the present invention, the mutation comprises an amino acid modification which enhances immune activation thereof by the movement protein (MP) of the ToBRFV as compared to the wild-type Tm-2² protein.

According to embodiments of the present invention, the Tm-2² protein comprises an amino acid modification which enhances immune activation thereof by the movement protein (MP) of the ToBRFV as compared to the wild-type Tm-2² protein.

According to embodiments of the present invention, the amino acid modification is in the leucine rich repeat (LRR) domain of the Tm-2² protein.

According to embodiments of the present invention, the Tm-2² protein comprises an amino acid modification at any one of positions 528, 604 or 652, as compared to the wild-type Tm-2² protein.

According to embodiments of the present invention, the modification is a substitution.

According to embodiments of the present invention, the modification at position 528 is F528S.

According to embodiments of the present invention, the modification at position 604 is S604N.

According to embodiments of the present invention, the modification at position 652 is I652M.

According to embodiments of the present invention, the tomato plant is resistant to tomato mosaic virus (ToMV) and tobacco mosaic virus (TMV).

According to embodiments of the present invention, the plant part is selected from the group consisting of roots, stems, leaves, cotyledons, flowers, fruit, embryos and pollen.

According to embodiments of the present invention, the crossing comprising pollinating.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced. In the drawings:

FIGS. 1A-H. MP^{ToBRFV} overcomes Tm-2² resistance in tomato. (A) An infectious clone of ToMV (top) compared to a recombinant version of ToMV with MP^{ToBRFV} replacing its native MP (ToMV^{MP-ToBRFV} ); bottom). Segmented lines mark the replaced region. (B-D) Representing leaves from tm-2/tm-2 tomato plants (CV. Moneymaker): (B) Non-infected plants, (C) ToMV-infected plants and (D) ToMV ToMV-MPMP^{ToBRFV}. (B-D) Representing leaves from tm-2/tm-2 tomato plants (CV. Moneymaker): (B) Non-infected plants, (C) ToMV-infected plants and (D) ToMV^{MP-ToBRFV}-infected plants. (E-G) Representing leaves from Tm-2²/Tm-2² tomato plants (CV. Moneymaker): (E) Non-infected plants, (F) ToMV-infected plants and (G) ToMV^{MP-ToBRFV} -infected plants. The 3^{rd} emerged leaf from the apex was sampled for each treatment. (H) A table summary of the number of symptomatic and infected plants of each treatment. Scale bars=5 cm.

FIGS. 2A-C. MP^{ToBRFV} overcomes Tm-2² resistance in N. benthamiana. (A) An infectious clone of TMV-GFP (top) compared to a recombinant version of TMV-GFP with MP^{ToBRFV} replacing its native MP (TMV-GFP^{MP-ToBRFV} ); bottom). Segmented lines mark the replaced region. (B) N. benthamiana plants infected with TMV-GFP alone (left) or with the expression of p35S: Tm-2² (right). (C) N. benthamiana plants infected with TMV-GFP^{MP-ToBRFV} alone (left) or with the expression of p35S:Tm-2² (right).

FIGS. 3A-F. Overexpression of MP^{ToBRFV} does not trigger Tm-2²-mediated cell death in tomato and N. benthamiana. (A-C) Transient expression of MP^{TMV} (A), MP^{ToBRFV} (B) and empty vector in tomato cv. Ikram containing the Tm-2² resistance gene. (D-E) Transient expression of MP^{TMV} (D) and MP^{ToBRFV} (E) in N. benthamiana leaves with or without Tm-2². (F) Transient expression of Tm-2² in N. benthamiana leaves served as negative control.

FIG. 4. Library preparation and screening process of Tm-2² mutant clones. The Tm-2² ORF was amplified using two separate PCR reactions: high fidelity PCR for the CC-NB region and error prone PCR to generate mutations in the LRR part of the gene. Both PCR products were assembled in a golden-gate level 0 cassette. The resulting colonies were pooled together and ORF was inserted into a level 2 plant expression plasmid. After an additional cycle of pooling, the resulting level 2 plasmids were transformed to agrobacterium and colonies were isolated to form the library. Mutant Tm-2² clones were screened by their co-expression with MP^{ToBRFV} followed by detection of necrotic lesions indicative of HR.

FIGS. 5A-C. Isolation of Tm-2² mutant clones whose products recognize MP^{ToBRFV} using directed evolution. (A) Isolation of ten different Tm-2² mutant clones whose expression triggers HR in response to MP^{ToBRFV}. (B) A table describing the number of clone, intensity of HR in response MP^{ToBRFV} (+ mild, ++ moderate, +++ severe), and locations of the different mutations. A silent mutation is defined as a synonymous mutation that does not alter amino acid identity (C) Schematic representation of the Tm-2² protein with amino acids that were found to be changed in at least two of the MP^{ToBRFV} recognizing clones (I528, S604 and I652).

FIGS. 6A-D. Validation of directed-evolution Tm-2² mutations that confer MP^{ToBRFV} recognition. Transient expression of Tm-2² containing each of the following mutations: F528S (A), S604N (B) and I652M (C). Tm-2² mutant clones were either expressed alone (left) or co-expressed with MP^{ToBRFV} (right). (D) Co-expression of non-mutant Tm-2² with MP^{TMV} or MP^{ToBRFV} served as positive and negative control, respectively.

FIGS. 7A-F. Transient expression of the Tm-2² variants confers resistance against a resistance-breaking TMV-GFP. Infection with a resistance-breaking TMV-GFP vector containing MP^{ToBRFV} (TMV-GFP^{MP-ToBRFV} ) as a replacement for its native MP. Virus vector was expressed in N. benthamiana leaf alone (A), or co-expressed with native Tm-2² (B), Tm-2² F528S (C), Tm-2² S604N (D) and Tm-2² I652M (E). Left panel—GFP fluorescence in the infected leaf, middle panel—GFP fluorescence of the whole plant, indicating systemic infection and right panel—viral symptoms. Images were taken 6 days after infection. (F) Quantification of GFP fluorescence in the 6^{th} leaf from the infection site.

FIG. 8. Predicted structural changes in the isolated Tm-2² mutants. Homology-based models of the Tm-2² and the Tm-2² mutants using ZAR1 structure (Wang et al., 2019). Small image—the structure of the whole Tm-2² protein. Large image—alignment of the Tm-2² LRR domains of the different Tm-2² variants: white—non-mutant Tm-2², green—Tm-2² F538S, purple—Tm-2² S604N, yellow—Tm-2² I652M. Red arrows indicate the mutated sites in the structure. White arrows indicate loop regions in which structural changes occur as compare to the wild-type Tm-2².

### DESCRIPTION OF SPECIFIC EMBODIMENTS OF THE INVENTION

The present invention, in some embodiments thereof, relates to tomato plants which are resistant to the deleterious effects of tomato brown rugose fruit virus (ToBRFV).

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

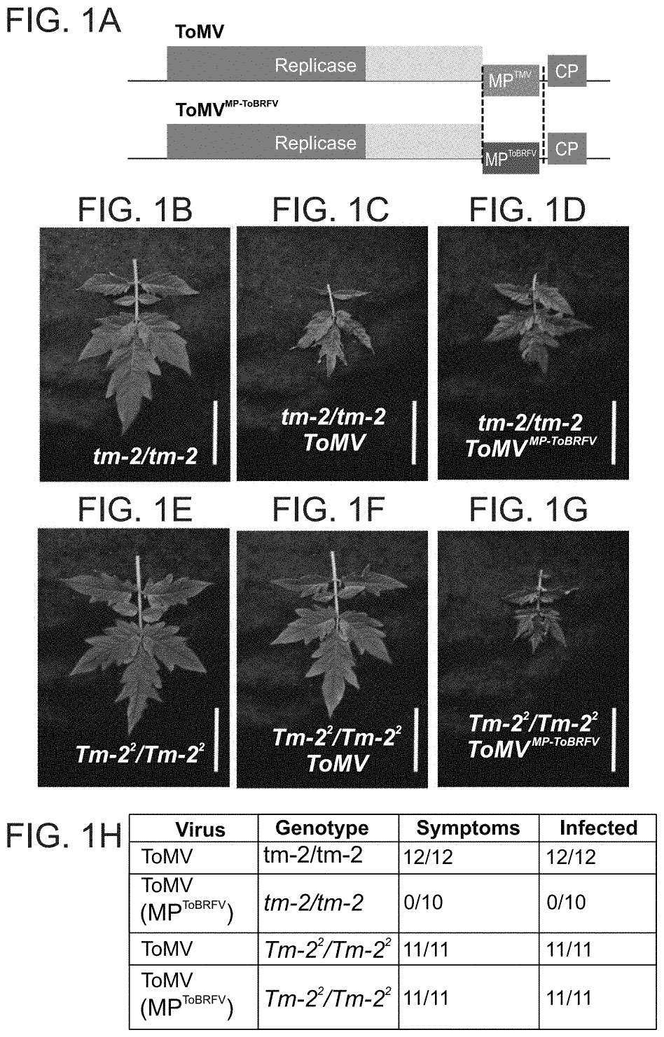

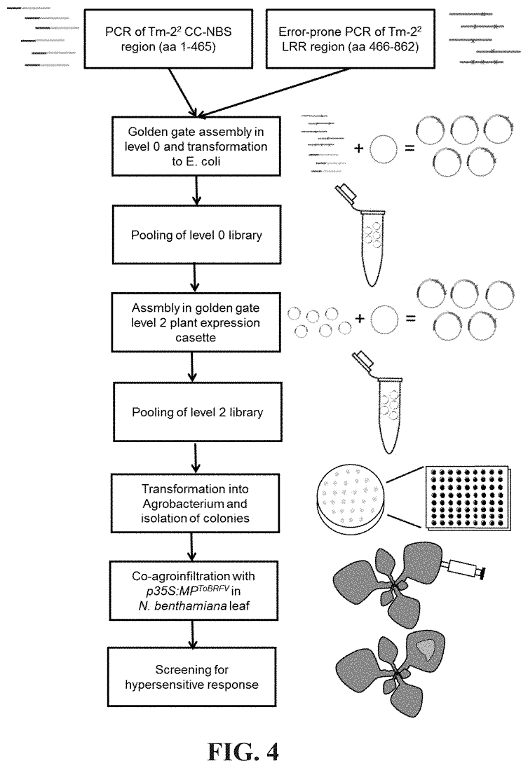

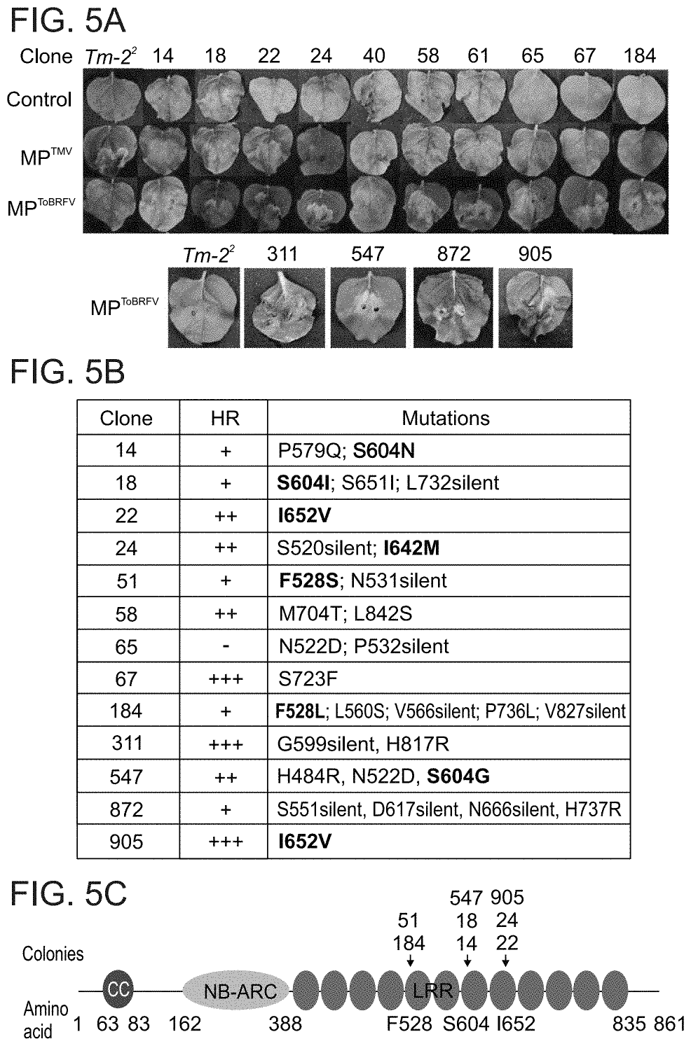

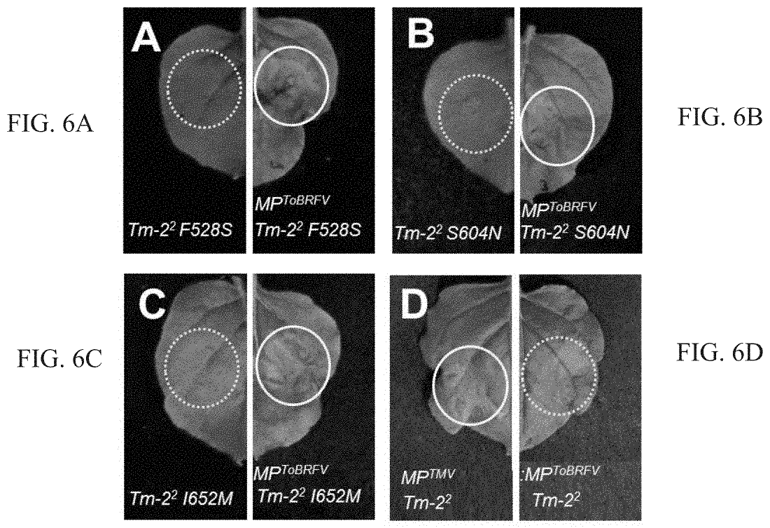

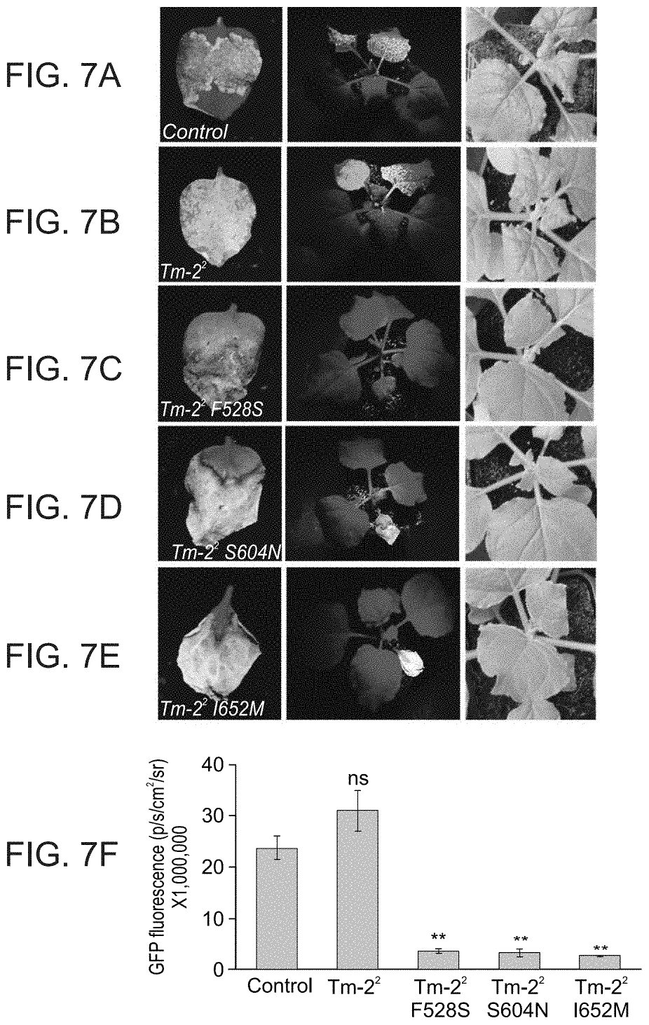

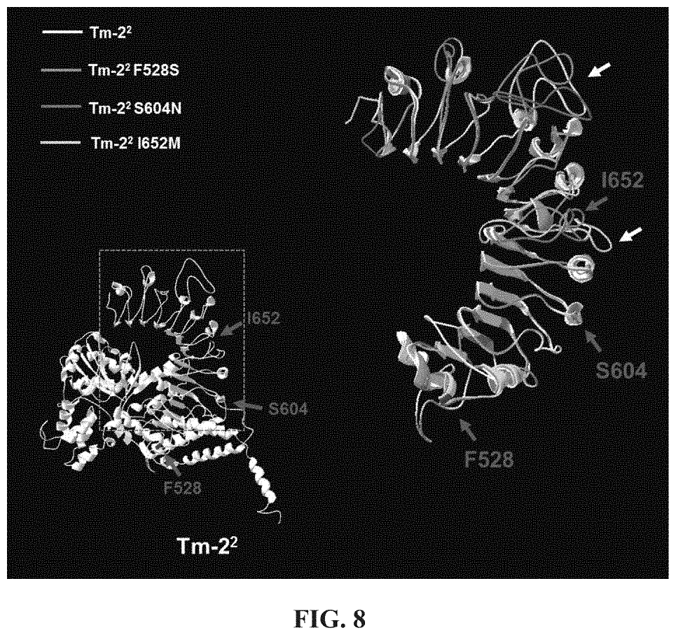

ToBRFV is an emerging and devastating Tobamovirus causing substantial damage to tomato crops around the world. The main cause for the ToBRFV epidemic is that it overcomes all genetic resistances in tomato, including the Tm-2² resistance gene, which has been durable against tobamoviruses for over than 50 years. The present inventors have now established that the cause for Tm-2² resistance breaking by ToBRFV is the lack of MP^{ToBRFV} recognition (FIGS. 1A-11 and FIG. 2A-C). A directed evolution approach was used to modify the Tm-2² gene so it recognized MP^{ToBRFV.} Based on the appearance of necrosis in N. benthamiana leaves (FIGS 1B-F), the present inventors established a screening system for Tm-2² for the identification of mutations that confer MP^{ToBRFV} recognition. A Tm-2² mutant library was constructed (FIG. 4) and screening yielded eight individual Tm-2² clones able to recognize MP^{ToBRFV} (FIGS. 5A-B). These clones enabled the identification of three single-nucleotide mutations, which confer MP^{ToBRFV} recognition (FIGS. 6A-D) and resistance against a resistance-breaking done of TMV-GFP expressing MP^{ToBRFV} (FIGS. 7A-F). All three mutations were located on the convex side of the Tm-2² LRR, suggesting that, this region plays a role in MP recognition specificity (FIG. 8).

Taken together, these results pave the way for new tomato lines which are resistant to the devastating effects of ToBRFV.

Thus, according to a first aspect of the present invention, there is provided a tomato plant or a part thereof, expressing a Tm-2² protein having an amino acid sequence which renders the plant resistant to tomato brown rugose fruit virus (ToBRFV).

The term “plant” as used herein encompasses whole plants, a grafted plant, ancestors and progeny of the plants and plant parts, including seeds, shoots, stems, roots, rootstock, scion, and plant cells, tissues and organs. The plant may be in any form including suspension cultures, embryos, meristematic regions, callus tissue, leaves, gametophytes, sporophytes, pollen, and micro spores.

The tomato plant can be of a cultivated genetic background or a wild tomato genetic background.

As used herein, the term “tomato” refers to a plant, line or population within the species Solanum lycopersicum (synonyms are Lycopersicon lycopersicum or Lycopersicon esculentum) or formerly known under the genus name of Lycopersicon including but not limited to L. cerasiforrne, L. cheesmanii, L. chilense, L. chmielewskii, L. esculentum (now S. pennellii), L. hirsutum, L. parviborum, L. pennellii, L. peruvianum, L. pimpinellifolium, or S. lycopersicoides. The newly proposed scientific name for L. esculentum is S. pennellii. Similarly, the names of the wild species may be altered. L. pennellii has become S. pennellii, L. hirsutum may become S. habrochaites, L. peruvianum may be split into S. ‘N peruvianum’ and S. ‘Callejon de Hueyles’, S. peruvianum, and S. corneliomuelleri, L. parviflorum may become S. neorickii, L. chmielewskii may become S. chmielewskii, L. chilense may become S. chilense, L. cheesmaniae may become S. cheesmaniae or S. galapagense, and L. pimpinellifolium may become S. pimpinellifolium.

Generally a cultivated tomato refers to tomato which is suitable for consumption and meets the requirements for commercial cultivation, e.g. typically classified as Solanum lycopersicum. In addition to the tomato plants themselves, and the parts thereof suitable for consumption, such as the fruit, the invention comprises parts or derivatives of the plant suitable for propagation. Examples of parts suitable for propagation are organ tissues, such as leaves, stems, roots, shoots and the like, protoplasts, somatic embryos, anthers, petioles, cells in culture and the like. Derivatives suitable for propagation are for instance seeds. The plants according to the invention can be cultivated or propagated in the conventional manner but also by means of tissue culture techniques from plant parts.

The present invention is aimed at using any tomato cultivars, such as of domestic use, fresh market tomatoes and processing tomatoes.

The choice of the variety depends on market demand, regional adaptability, disease resistance and the end use of the product. Exemplary segments for fresh market tomatoes include, but are not limited to, Beef (fruit weight of about 220-400 gr), Standard (fruit weight of about 160-220 gr) and Cluster (uniform fruit weight of about 120-180 gr). Such varieties are available from major seed companies e.g., Grodena, Macarena, Estatio, Zouk, Climbo and Climstar, all available from Syngenta. Other varieties can be proprietary or available from other vendors, including but not limited to, Cherry-micro (up to 5 gr) round cherry, mini round cherry (7.5-15 gr), mini plum elongated cherry (10-25 gr). Examples for these varieties are: Creativo (Clause), Batico (Nirit seeds), Shiren (Hazera Genetics). Cocktail round and elongated (25-40 gr): Romanita, Cherry and Cocktail with red, yellow, orange, pink, zebra, chocolate background. Examples include, but are not limited to, Summer sun (Hazera Genetics), Black pearl (Burpee) Tyty (Tomodori). Roma determinate and indeterminate. 120-200 gr. Examples for the intermediate marker include, but are not limited to, lancelot (Vilomorin) and Parsifal (Vilomorin). Pink tomato divided to beef (220-400), standard (160-220) and cluster (120-180). Example: Momotaro type, Cor di bue tomato, (150-350 gr), Pinton (250-300 gr), open field tomato-determinate or semi-determinate (180-400 gr).

Exemplary cultivars of processing tomatoes include, but are not limited to, Roma, SUN 6366, AB 2, Heinz 9780, Heinz 9557, Halley 3155 and Hypeel 303.

There are two major types of tomato growth: determinate and indeterminate. Determinate growth produces “bush” tomatoes and which are bred for compactness. The entire plant stops growing once the terminal fruit ripens, the remainder of the fruit all ripen nearly simultaneously, and then the plant dies. Indeterminate growth produces tomatoes that can grow up to 10 feet in height (so-called “vining” tomatoes) and will only stop growing when killed (e.g. by frost). Their fruits ripen sequentially. In a typical plant, all growth arises from the reiteration of modular sympodial units that each produce three leaves and a multiflowered inflorescence. Most field-grown varieties of tomato, including M82, are determinate plants whose shoots produce an average of six sympodial units, each harboring a single inflorescence, within which leaf number gradually decreases before a precocious termination of growth. In general, determinate tomatoes are suitable for open field production. Semi-determinate and indeterminate “cultivated” varieties are suitable for staked cultivation in the open field or protected nets and for glasshouse cultivation.

According to an embodiment of the invention the tomato plant is a determinate tomato.

According to an embodiment of the invention the tomato plant is an indeterminate tomato.

According to an embodiment of the invention the tomato plant is a semi-determinate tomato.

According to an embodiment, the tomato is selected from the group consisting of a single fruit per truss, branched tomato and cherry tomato.

The tomato plant of this aspect of the present invention has an increased resistance to tomato brown rugose fruit virus (ToBRFV) as compared to that of a control tomato plant of the same genetic background not expressing the modified Tm-2² protein.

The “same genetic background” refers to at least 95%, 96%, 97%, 98%, 99% or 99.9% of the genome is shared between the plant and the non-modified plant.

As used herein “increased resistance” refers to at least 10%, 20%, 30%, 40%, 50%, 60%, 70%, 80%, 85%, 90% or even 95%, increase in viral resistance as compared to that of a tomato plant of the same genetic background not expressing the modified Tm-2² protein and as manifested by either delayed or milder symptoms appearance or reduced accumulation of RNA of the virus, as assayed by methods which are well known in the art (see Examples section which follows).

According to a specific embodiment, increased resistance is evidenced for at least 10 days, 20 days, 30 days or longer.

The term “Tm-2²” refers to a receptor which confers resistance to tobamoviruses in the tomato plant. An exemplary amino acid sequence of wild-type Tm-2² is set forth in SEQ ID NO: 25. An exemplary nucleic acid sequence which encodes wild-type Tm-2² is set forth in SEQ ID NO: 26.

In one embodiment, the plant is homozygotic for a Tm-2² mutation, which renders the plant resistant to tomato brown rugose fruit virus (ToBRFV). In another embodiment, the plant is heterozygotic for the Tm-2² mutation.

Tomato plants of this aspect of the present invention may be characterized by having both alleles of the Tm-2² gene having a mutation that results in enhanced resistance to ToBRFV. In this case, the Tm-2² may be in a homozygous form or in a heterozygous form. According to this embodiment, homozygosity is a condition where both alleles at the Tm-2² locus are characterized by the same nucleotide sequence. Heterozygosity refers to different conditions of the gene at the Tm-2² locus.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

The term “gene” as used herein refers to an inherited factor that determines a biological characteristic of an organism (i.e. a tomato plant), an “allele” is an individual gene in the gene pair present in the (diploid) tomato plant.

A plant is called “homozygous” for a gene when it contains the same alleles of said gene, and “heterozygous” for a gene when it contains two different alleles of said gene. The use of capital letters indicates a dominant (form of a) gene and the use of small letters denotes a recessive gene: “X,X” therefore denotes a homozygote dominant genotype for gene or property X; “X,x” and “x,X” denote heterozygote genotypes; and “x,x” denotes a homozygote recessive genotype. As commonly known, only the homozygote recessive genotype will generally provide the corresponding recessive phenotype (i.e. lead to a plant that shows the property or trait “x”) whereas the heterozygotic and homozygote dominant genotypes will generally provide the corresponding dominant phenotype (i.e. lead to a plant that shows the property or trait “X”), unless other genes and/or factors such as multiple alleles, suppressors, codominance etc. (also) play a role in determining the phenotype.

In one embodiment, the genome of the tomato plant comprises a nucleic acid sequence encoding a mutated Tm-2² protein (as compared to wild-type Tm-2²) which brings about enhanced resistance to ToBRFV.

The mutation may be an insertion, a deletion or a substitution.

The mutation may be referred to as a gain or alteration of function mutation—i.e. the mutation is responsible for acquiring resistance to ToBRFV.

The Tm-2² protein of this aspect of the present invention may comprise a single mutation, two mutations, three mutations or more as compared to the wild-type Tm-2² protein.

Preferably, the mutation allows for activation of the Tm-2² protein by the movement protein (MP) of the ToBRFV.

Activation of the Tm-2² receptor typically brings about self-association of the Tm-2² protein that allows the immune signal to occur.

In one embodiment, the mutation enhances binding of the Tm-2² receptor to the movement protein (MP) of the ToBRFV as compared to the wild-type Tm-2² protein.

Preferably, the amino acid modification is in the leucine rich repeat (LRR) region of the Tm-2² receptor (between amino acid 388 and amino acid 861.

Thus, for example the present invention contemplates plants expressing Tm-2² receptors which are at least 90% identical to the amino acid sequence as set forth in SEQ ID NO: 25 and having an amino acid modification at any one of positions 528, 604 and/or 652, as compared to the wild-type Tm-2² protein.

The “percent identity” of two amino acid sequences may be determined using the algorithm of Karlin and Altschul, Proc. Natl. Acad. Sci. USA 87:2264-68, 1990, modified as in Karlin and Altschul, Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, word length=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25: 3389 3402. Alternatively, PSI BLAST or PHI BLAST can be used to perform an iterated search which detects distant relationships between molecules (Id.). When utilizing BLAST, Gapped BLAST, PSI Blast and PHI Blast programs, the default parameters of the respective programs (e.g., of XBLAST and NBLAST) can be used (see, e.g., National Center for Biotechnology Information (NCBI) on the worldwide web, ncbi(dot)nlm(dot)nih(dot)gov). Another specific, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps, such that any software for protein sequence alignment can be used. In calculating percent identity, typically only exact matches are counted.

In one embodiment, the modification at position 528 is a F528S substitution.

In one embodiment, the modification at position 604 is a S604N substitution.

In one embodiment, the modification at position 652 is an I652M substitution.

Additional contemplated mutations include P579Q, S604I, S651I, M704T, L842S, N522D, S723F, L560S, N522D, H737R, I652V, H817R and P736L. Other mutations are summarized in FIG. 5B herein.

In one embodiment, the modification does not affect the resistance of the Tm-2² to other viruses of the Tobamovirus genus, such as tomato mosaic virus (ToMV) and/or tobacco mosaic virus (TMV).

In another embodiment, the modification lowers the resistance of the Tm-2² to other viruses of the Tobamovirus genus, such as tomato mosaic virus (ToMV) and/or tobacco mosaic virus (TMV). For this embodiment, it is envisaged that only one of the alleles comprises the mutations that increase resistance to ToBRFV, whilst the other allele does not comprise these mutations such that this allele confers resistance to ToMV and TMV.

The present inventors contemplate both chemical mutagenesis and recombinant techniques for the generation of the tomato plants of the present invention.

Thus, the tomato plants of the present invention may be generated by exposing the tomato plant or part thereof to a chemical mutagen. Examples of chemical mutagens include, but are not limited to nitrous acid, alkylating agents such as ethyl methanesulfonate (EMS), methyl methane sulfonate (MMS), diethylsulfate (DES), and base analogs such as 5-bromo-deoxyuridine (5BU). In such a case the plant is non-genetically modified with an agent for inducing mutations in Tm-2².

Initial exposure is typically followed by additional steps of selfing, selection, crossing and selfing or combinations thereof, where any step can be repeated more than once, as long as the gain-of-function in the Tm-2² gene (i.e. acquired resistance) is present. Selection can be phenotypic or using marker-assisted breeding as further described hereinbelow.

According to another specific embodiment, the non-genetically modified plant of the invention results from a spontaneous genetic event incurred by multiple crossings/selfings.

Any of the below methods can be directed to any part of the Tm-2² gene as long as a gain-of-function is achieved (acquired resistance). In specific embodiments, the agent is directed to the leucine rich repeat (LRR) domain of said Tm-2².

When needed further steps of selfing are effected in order to achieve a homozygous form of the mutation.

As used herein “target sequence” refers to the Tm-2² DNA coding or RNA transcript.

Genome Editing using engineered endonucleases - this approach refers to a reverse genetics method using artificially engineered nucleases to cut and create specific double-stranded breaks at a desired location(s) in the genome, which are then repaired by cellular endogenous processes such as, homology directed repair (HDR) and non-homologous end-joining (NHEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, MT et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically, a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the Fokl domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, CA).

Method for designing and obtaining TALENs are described in e.g. Reyon et al. Nature Biotechnology 2012 May;30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, CA).

Another agent capable of downregulating AGL6 is a RNA-guided endonuclease technology e.g. CRISPR system (that is exemplified in great details in the Examples section which follows).

As used herein, the term “CRISPR system” also known as Clustered Regularly Interspaced Short Palindromic Repeats refers collectively to transcripts and other elements involved in the expression of or directing the activity of CRISPR-associated genes, including sequences encoding a Cas9 gene (e.g. CRISPR-associated endonuclease 9), a tracr (trans-activating CRISPR) sequence (e.g. tracrRNA or an active partial tracrRNA), a tracr-mate sequence (encompassing a “direct repeat” and a tracrRNA-processed partial direct repeat) or a guide sequence (also referred to as a “spacer”) including but not limited to a crRNA sequence (i.e. an endogenous bacterial RNA that confers target specificity yet requires tracrRNA to bind to Cas) or a sgRNA sequence (i.e. single guide RNA).

In some embodiments, one or more elements of a CRISPR system is derived from a type I, type II, or type III CRISPR system. In some embodiments, one or more elements of a CRISPR system (e.g. Cas) is derived from a particular organism comprising an endogenous CRISPR system, such as Streptococcus pyogenes, Neisseria meningitides, Streptococcus thermophilus or Treponema denticola.

In general, a CRISPR system is characterized by elements that promote the formation of a CRISPR complex at the site of a target sequence (also referred to as a protospacer in the context of an endogenous CRISPR system).

In the context of formation of a CRISPR complex, “target sequence” in this case AGL6 refers to a sequence to which a guide sequence (i.e. guide RNA e.g. sgRNA or crRNA) is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. Thus, according to some embodiments, global homology to the target sequence may be of 50%, 60%, 70%, 75%, 80%, 85%, 90%, 95% or 99%. A target sequence may comprise any polynucleotide, such as DNA or RNA polynucleotides. In some embodiments, a target sequence is located in the nucleus or cytoplasm of a cell.

Thus, the CRISPR system comprises two distinct components, a guide RNA (gRNA) that hybridizes with the target sequence, and a nuclease (e.g. Type-II Cas9 protein), wherein the gRNA targets the target sequence and the nuclease (e.g. Cas9 protein) cleaves the target sequence. The guide RNA may comprise a combination of an endogenous bacterial crRNA and tracrRNA, i.e. the gRNA combines the targeting specificity of the crRNA with the scaffolding properties of the tracrRNA (required for Cas9 binding). Alternatively, the guide RNA may be a single guide RNA capable of directly binding Cas.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence.

In some embodiments, the tracr sequence has sufficient complementarity to a tracr mate sequence to hybridize and participate in formation of a CRISPR complex. As with the target sequence, a complete complementarity is not needed, provided there is sufficient to be functional. In some embodiments, the tracr sequence has at least 50%, 60%, 70%, 80%, 90%, 95% or 99% of sequence complementarity along the length of the tracr mate sequence when optimally aligned.

Introducing CRISPR/Cas into a cell may be effected using one or more vectors driving expression of one or more elements of a CRISPR system such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. A single promoter may drive expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron).

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, transformed into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After transformation and positive selection, homologously targeted clones are identified. Next, a second targeting vector that contains a region of homology with the desired mutation is transformed into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRY”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

As mentioned, the tomato plant of the present invention may also be generated using other techniques including but not limited to genome editing of Tm-2² gene.

Thus, for example, gene knock-in or gene knock-out constructs including sequences homologous with the Tm-2² gene can be generated and used to insert an ancillary sequence into the coding sequence of the enzyme encoding gene, to thereby modify this gene.

These construct preferably include positive and negative selection markers and may therefore be employed for selecting for homologous recombination events. One ordinarily skilled in the art can readily design a knock-in/knock-out construct including both positive and negative selection genes for efficiently selecting transformed plant cells that underwent a homologous recombination event with the construct. Such cells can then be grown into full plants. Standard methods known in the art can be used for implementing knock-in/knock out procedure. Such methods are set forth in, for example, U.S. Pat. Nos. 5,487,992, 5,464,764, 5,387,742, 5,360,735, 5,347,075, 5,298,422, 5,288,846, 5,221,778, 5,175,385, 5,175,384, 5,175,383, 4,736,866 as well as Burke and Olson, Methods in Enzymology, 194:251-270, 1991; Capecchi, Science 244:1288-1292, 1989; Davies et al., Nucleic Acids Research, 20 (11) 2693-2698, 1992; Dickinson et al., Human Molecular Genetics, 2(8):1299-1302, 1993; Duff and Lincoln, “Insertion of a pathogenic mutation into a yeast artificial chromosome containing the human APP gene and expression in ES cells”, Research Advances in Alzheimer's Disease and Related Disorders, 1995; Huxley et al., Genomics, 9:742-750 1991; Jakobovits et al., Nature, 362:255-261 1993; Lamb et al., Nature Genetics, 5: 22-29, 1993; Pearson and Choi, Proc. Natl. Acad. Sci. USA, 1993, 90:10578-82; Rothstein, Methods in Enzymology, 194:281-301, 1991; Schedl et al., Nature, 362: 258-261, 1993; Strauss et al., Science, 259:1904-1907, 1993, WO 94/23049, WO93/14200, WO 94/06908 and WO 94/28123 also provide information.

According to a specific embodiment, the plant is a transgenic plant (e.g., for a genome editing agent as described herein below).

According to a specific embodiment, the plant may be a transgenic plant but the transgene may not be associated with (i.e., not the cause for) resistance to ToBRFV, as described herein. For example, the transgene may function to improve biotic stress resistance, pesticide resistance or abiotic stress resistance.

According to a particular embodiment of the present invention, the tomato plant is generated by introduction thereto of a nucleic acid construct, the nucleic acid construct comprising a nucleic acid sequence encoding a polynucleotide agent which up-regulates an expression of Tm-2² having a mutation which brings about an enhanced resistance to ToBRFV and a cis-acting regulatory element capable of directing an expression of the polynucleotide agent in the plant.

Constructs useful in the methods according to the present invention may be constructed using recombinant DNA technology well known to persons skilled in the art. The gene constructs may be inserted into vectors, which may be commercially available, suitable for transforming into plants and suitable for expression of the gene of interest in the transformed cells. The genetic construct can be an expression vector wherein the nucleic acid sequence is operably linked to one or more regulatory sequences allowing expression in the plant cells.

The polynucleotide according to this aspect of the present invention may encode Tm-2² having for example an F528S mutation, an S604N mutation and/or an I652M. The polypeptide sequence of an exemplary Tm-2² having the above described mutation is typically at least 90% homologous, at least 91% homologous, at least 92% homologous, at least 93% homologous, at least 94% homologous, at least 95% homologous, at least 96% homologous, at least 97% homologous, at least 98% homologous, at least 99% homologous, or 100% homologous to the sequence set forth in SEQ ID NO: 25. The nucleic acid sequence of an exemplary polynucleotide which encodes such a protein may be at least 90% homologous, at least 91% homologous, at least 92% homologous, at least 93% homologous, at least 94% homologous, at least 95% homologous, at least 96% homologous, at least 97% homologous, at least 98% homologous, at least 99% homologous, or 100% homologous to the nucleic acid sequence set forth in SEQ ID NO: 26.

In a particular embodiment of the present invention the regulatory sequence is a plant-expressible promoter.

As used herein the phrase “plant-expressible” refers to a promoter sequence, including any additional regulatory elements added thereto or contained therein, is at least capable of inducing, conferring, activating or enhancing expression in a melon cell, tissue or organ.

The promoter may be a regulatable promoter, a constitutive promoter or a tissue-associated promoter.

As used herein, the term “regulatable promoter” refers to any promoter whose activity is affected by specific environmental or developmental conditions.

As used herein, the term “constitutive promoter” refers to any promoter that directs RNA production in many or all tissues of a plant transformant at most times.

As used herein, the term “tissue-associated promoter” refers to any promoter which directs RNA synthesis at higher levels in particular types of cells and tissues (e.g., a fruit-associated promoter).

Exemplary promoters that can be used to express an operably linked nucleic acid sequence (i.e. transgene) include the cauliflower mosaic virus promoter, CaMV and the tobacco mosaic virus, TMV, promoter.

Other promoters that can be used in the context of the present invention include those described in U.S. Patent No. 20060168699 and by Hector G. Numez-Palenius et al. [Critical Reviews in Biotechnology, Volume 28, Issue 1 March 2008, pages 13 — 55], both of which are incorporated herein by reference.

Plant cells may be transformed stably or transiently with the nucleic acid constructs of the present invention. In stable transformation, the nucleic acid molecule of the present invention is integrated into the plant genome and as such it represents a stable and inherited trait. In transient transformation, the nucleic acid molecule is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276).

The principle methods of causing stable integration of exogenous DNA into plant genomic DNA include two main approaches:
(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.
(ii) Direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledenous plants.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by the present invention.

Transient transformation can be effected by any of the direct DNA transfer methods described above or by viral infection using modified plant viruses.

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Regardless of the method used to produce the tomato plant of some embodiments of the invention, once plants or any reproductive material is at hand, it is selected for the ToBRFV resistance trait.

Thus, according to an aspect of the invention there is provided a method of selecting a tomato plant being resistant to ToBRFV, the method comprising detecting in a genome of a tomato plant a gain of function mutation in the Tm-2² gene, wherein presence of the mutation is indicative of a tomato plant being resistant.

Many methods are known in the art for analyzing for mutations including for example single base extension (SBE), allele-specific primer extension sequencing (ASPE), DNA sequencing, RNA sequencing, microarray-based analyses, universal PCR, Melting Curve SNP method, allele specific extension, hybridization, mass spectrometry, ligation, extension-ligation, Flap Endonuclease-mediated assays, restriction fragment length polymorphism (RFLP), electrophoresis, sequence alignment, allelic specific oligonucleotide hybridization (ASO) and random amplified polymorphic DNA (RAPD).

Thus, the present invention contemplates oligonucleotides (e.g. primers) that can be used to distinguish between the mutated and non-mutated form of Tm-2² gene.

Thus, once a plant carrying the gain of function genetic alteration is identified it is considered as being resistant to ToBRFV. This plant material can be used as a breeding material in the development of tomato varieties having agriculturally desired traits.

According to one embodiment, the plants of the present invention are of a hybrid variety—i.e. are generated following the crossing (i.e. mating) of two non-isogenic plants both being homozygous for a gain of function mutation in the Tm-2² gene. The hybrid may be an F₁ Hybrid.

An “F₁ Hybrid” as used herein, refers to first generation progeny of the cross of two non-isogenic plants.

The development of tomato hybrids of the present invention requires the development of stable parental lines. In breeding programs desirable traits from two or more germplasm sources or gene pools are combined to develop superior breeding varieties. Desirable inbred or parent lines are developed by continuous self-pollinations and/or backcrosses and selection of the best breeding lines, sometimes utilizing molecular markers to speed up the selection process.

Once the parental lines that give the best hybrid performance have been identified e.g., both carrying the gain of function mutation as described above e.g., in the Tm-2² gene, the hybrid seed can be produced indefinitely, as long as the homozygosity of the parents are maintained. According to one embodiment the tomato plants of the present invention are stable parent plant lines (carrying the gain of function mutation e.g., in the Tm-2² gene in a heterozygous form or a homozygous form).

As defined herein, the phrase “stable parental lines” refers to open pollinated, inbred lines, stable for the desired plants over cycles of self-pollination and planting. According to a specific embodiment, 95% of the genome is in a homozygous form in the parental lines of the present invention.

A common practice in plant breeding is using the method of backcros sing to develop new varieties by single trait conversion.

The phrase “single trait conversion” as used herein refers to the incorporation of new single gene into a parent line wherein essentially all of the desired morphological and physiological characteristics of the parent lines are recovered in addition to the single gene transferred.

The term “backcrossing” as used herein refers to the repeated crossing of a hybrid progeny back to one of the tomato plants. The parental tomato plant which contributes the gene for the desired characteristic is termed the non-recurrent or donor parent. This terminology refers to the fact that the non-recurrent parent is used one time in the backcross protocol and therefore does not recur. The parental tomato plant to which the gene from the non-recurrent parent are transferred is known as the recurrent parent as it is used for several rounds in the backcrossing protocol.

In a typical backcross protocol, a plant from the original varieties of interest (recurrent parent) is crossed to a plant selected from second varieties (non-recurrent parent) that carries the single gene of interest to be transferred. The resulting progeny from this cross are then crossed again to the recurrent parent and the process is repeated until a tomato plant is obtained wherein essentially all of the desired morphological and physiological characteristics of the recurrent parent are recovered in the converted plant, in addition to the single transferred gene from the non-recurrent parent.

Thus, near-isogenic lines (NIL) may be created by many backcrosses to produce an array of individuals that are nearly identical in genetic composition except for the trait or genomic region under interrogation in this case gain of function genetic alteration e.g., in the Tm-2² gene.

Backcrossing methods can be used with the present invention to improve or introduce a characteristic into the parent lines. Marker assisted breeding (selection) as described above can be used in this method.

According to a specific embodiment, the plant or the plant seed is an inbred.

According to a specific embodiment, the plant is a hybrid plant or the seed is a hybrid seed.

The invention also relates to progeny of the tomato, eggplant, pepper plants of the invention. Such progeny can be produced by sexual or vegetative reproduction of a plant of the invention or a progeny plant thereof. The regenerated progeny plant grows fruits independent of fertilization in the same or a similar way as the parent. In addition to this, the progeny plant may be modified in one or more other characteristics. Such additional modifications are for example effected by mutagenesis or by transformation with a transgene.

As used herein the word “progeny” is intended to mean the offspring or the first and all further descendants from a cross with a plant of the invention that shows fertilization independent fruit formation. Progeny of the invention are descendants of any cross with a plant of the invention that carries the mutation (in a homozygous form) trait that leads to fertilization independent fruit formation.

“Progeny” also encompasses plants that carry the trait of the invention which are obtained from other plants of the invention by vegetative propagation or multiplication.

As mentioned, embodiments described herein, furthermore, relate to hybrid seed and to a method of producing hybrid seed comprising crossing a first parent plant with a second parent plant and harvesting the resultant hybrid seed. In this case the trait is recessive, therefore both parent plants need to be homozygous for the fertilization independent fruit formation trait in order for all of the hybrid seed to carry the trait of the invention. They need not necessarily be uniform for other traits.

Embodiments described herein also relate to the germplasm of the plants. The germplasm is constituted by all inherited characteristics of an organism and according to the invention encompasses at least the facultative fertilization independent fruit formation trait of the invention.

Embodiments described herein also relate to cells of the plants that show the facultative fertilization independent fruit formation trait. Each cell of such plants carries the genetic information (i.e., mutation in Tm-2²) that leads to the resistance to ToBRFV. The cell may be an individual cell or be part of a plant or plant part, such as the fruit.

The present teachings further relate to consumed products which comprise the genomic (DNA) information (i.e., mutation in Tm-2²) that leads to the resistance to ToBRFV.

Fruits of any of the plants described herein may be selected or qualified for fruit color, Brix, pH, sugars, organic acids and defect levels (insect damage, mold, etc.) at ripening or post harvest. For example, tomatoes are typically transported to a large processing facility, where they are collected and where they may subsequently be washed, typically using chlorinated water and rinsed using tap water and further selected to remove those that present defects (e.g., inadequate ripening, disease damage, molds etc.). Tomatoes may be stored (especially those exhibiting improved shelf-life as described above) or immediately sent to the consumer (fresh-market tomatoes). Processing tomatoes may be processed into a wide variety of products.

For juice or pulp production, the tomatoes may be subject to oven dehydration and are comminuted and macerated (disintegrated and broken) to obtain a pumpable mass. As will be clear to the skilled person these operations per se are known and common in the field of tomato processing and any adjustments to the method can be made in this regard without departing from the scope.

Methods for processing tomatoes and/or producing tomato-based compositions are well known in the art, see generally U.S. Pat. No. 6,924,420. Also reported are specific methods for preparing, for example, paste (U.S. Pat. No. 7,074,451), sterile paste (U.S. Pat. No. 4,206,239), puree (U.S. Pat. No. 4,556,576), sauce (U.S. Pat. No. 7,122,217), solidified sauce (U.S. Pat. No. 4,038,424), barbecue sauce (U.S. Pat. No. 6,869,634), salsa (U.S. Pat. No. 5,914,146), ketchup (U.S. Pat. No. 6,689,279), tomato fiber composition (U.S. Pat. No.

7,166,315) and dehydrated tomato-product (U.S. Pat. No. 5,035,909). Methods of modifying the texture and consistency of tomato paste, pulp, and puree has also been reported, see, for example, U.S. Pat. No. 6,720,019.

Also provided is an edible processed tomato product comprising the tomato or an edible portion thereof (e.g., fruit or an edible part thereof).

Also provided is a tomato paste generated according to the present teachings. Examples of such edible products include, but are not limited to, canned tomatoes (whole), a tomato paste, a ketchup, a tomato sauce a tomato soup, a dehydrated tomato, a tomato juice, a tomato powder, a tomato dice, a crushed tomato, a chopped tomato and a tomato concentrate.

According to some embodiments, the products comprise the DNA (carrying a gain of function mutation in the Tm-2² that leads to the resistance to ToBRFV) of the tomato (e.g., paste, dried fruit, juice and the like).

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof. Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Plant Materials: Nicotiana banthamiana and Solanum lvcopersicum L. cv. Ikram (Syngenta) plants were grown at 25° C. under long day conditions. Well-developed leaves of five-week old plants were used for agro-infiltration.

Agro-infiltration for transient expression and N. benthamiana infection: A. tumefaciens strain EHA105 containing a binary vector were grown overnight at 28° C. Cell cultures were resuspended in MES buffer (10 mM MgCl2, 10 mM MES, 150 μM acetosyringone, pH 5.6) to an optical density of OD600=0.5. Bacterial cells containing the p35S:MP and p35s:Tm-2² vectors were either expressed alone or mixed at a 1:1 ratio and infiltrated into the abaxial side of the 4th or 5th leaf of N. benthamiana or Solanum lycopersicum.

Directed evolution library construction: All Golden Gate vectors were supplied by Addgene. The Tm-2² (AF536201) gene was cloned into pICH41308, a Level 0 CDS1 Golden Gate vector (Weber et al., 2011). Internal BsaI site was removed (sequence domestication) by one-step cloning using primers 60, 63 and 62, 61 (Table 1). The gene was then divided into two sections, each flanked by Bpil sites and sharing a 4-base sequence that would serve as a fusion site: the sequence coding for the CC-NBS regions at amino acids 1-470 (1,409 bp) was amplified by PCRBIO HiFi Polymerase (Cat#PB10.41-10), (primers 60, 99, Table 1). The sequence coding for the LRR (bp 1,405-2,586) served as a template for random mutagenesis, as described by Xu et al (1999). Briefly, 100 ng of template DNA was first amplified with addition of 40 μM Mn2+ to a PCR reaction that was carried out with Taq Ready Mix (HyLabs Cat. #EZ-3007). 2 μl of the product served as template for a second PCR, adding 40 μM of dITP to the reaction (primers 98, 61 for both reactions). The two sections of the Tm-2² gene were then inserted into the pICH41308 level 0 vector by Golden Gate assembly and transformed into DH5α cells. Colonies were then pooled together, plasmid DNA was extracted and cloned into the Golden Gate Level 2 binary vector pICH486988. Colonies were pooled again, and extracted DNA was transformed into A. tumefaciens. Individual A. tumefaciens clones were isolated, cataloged and frozen in −80° C. to form the Tm-2² mutant library.

TABLE 1

| # | Name | Sequence |
|---|---|---|
| 37 | MPswap1_ | ATAACCGGTCTCCATCATTGCAGG (SEQ ID NO: 1) |
| | AgeI_F | |
| 38 | MPswap2_R | ATATTGACTTTACCCTTAACAAAGCCATCTATAAACA (SEQ ID |
| | | NO: 2) |
| 39 | MPswap3_F | CTTTTTAGAAGTTTGTTTATAGATGGCTCTTGTTAAG (SEQ ID NO: |
| | | 3) |
| 40 | MPswap4_ | ATATTAATTAATGACAAGAACACGAACTGAGATGGAGTAGTGAT |
| | PacI_R | ACTGTAAGATCTATTTAATACGAATCTGAAT (SEQ ID NO: 4) |
| 41 | MPseqF | CCCTCCAGGTTCGTTTGTT (SEQ ID NO: 5) |
| 50 | TB_MP_specific_ | TTTGTTTGGTTTCGGCCTATTATT (SEQ ID NO: 6) |
| | 702_R | |
| 60 | Tm22_BpiI_F | TGAAGACATAATGGCTGAAATTCTTCTT (SEQ ID NO: 7) |
| 61 | Tm22stop_ | TGAAGACATAAGCTCATTTACTCAGCTTTTTA (SEQ ID NO: 8) |
| | BpiI R | |
| 62 | Tm22_BsaIde1_ | TGAAGACATACGATAGACATTGATCG (SEQ ID NO: 9) |
| | BpiI_F | |
| 63 | Tm22 BsaIde1_ | TGAAGACATTCGTCTCTAGACGTGTGAGC (SEQ ID NO: 10) |
| | BpiI_R | |
| 98 | Tm22 2552_ | TGAAGACATCATATAATGGAAGAATTTCAAG (SEQ ID NO: 11) |
| | DE_BpiI_F | |
| 99 | Tm22 2555 | TGAAGACATTATGTCCTTTTGGCAAGT (SEQ ID NO: 12) |
| | DE BpI_R | |
| 125 | Tm-22 S604N | GAAAACTGCCAAATAATATTGTCAAGCTC (SEQ ID NO: 13) |
| | F | |
| 126 | Tm-22 S604N | GAGCTTGACAATATTATTTGGCAGTTTTC (SEQ ID NO: 14) |
| | R | |
| 133 | Tm22 F528S | CATGATTGAGTTCTCCCGTTCAAATCCTA (SEQ ID NO: 15) |
| | F | |
| 134 | Tm22 F528S | TAGGATTTGAACGGGAGAACTCAATCATG (SEQ ID NO: 16) |
| | R | |
| 135 | Tm22 I652M | CAGTTGCTTTTCTATGAGCTCATTTTACCC (SEQ ID NO: 17) |
| | F | |
| 136 | Tm22 I652M | GGGTAAAATGAGCTCATAGAAAAGCAACTG (SEQ ID NO: 18) |
| | R | |
| 158 | ToMV KpnI F | ATGGTACCAGAGAAAGAGTGG (SEQ ID NO: 19) |
| 159 | ToMV Xma R | TTCCCGGGGATCCGTCGAC (SEQ ID NO: 20) |
| 160 | TB MP for | CTTAACAAGAGCCATCAAGAAATAAACTTC (SEQ ID NO: 21) |
| | ToMV R | |
| 161 | TB MP for | GAAGTTTATTTCTTGATGGCTCTTGTTAAG (SEQ ID NO: 22) |
| | ToMV F | |
| 162 | TB MPend for | ATTGAGTAAGACATATTTAATACGAATCTG(SEQ ID NO: 23) |
| | ToMV R | |
| 163 | TB MPend for | CAGATTCGTATTAAATATGTCTTACTCAAT (SEQ ID NO: 24) |
| | ToMV F | |

Site-directed mutagenesis: Site-directed mutagenesis was carried out on the Tm-2² gene in the Level-0 vector using 30 bp-long complementing ologonucleotides containing the mutation of interest, using PCRBIO HiFi Polymerase (Cat. #PB 10.41-10). Construction of ToMV^{MP-ToBRFV}: The vector pTLW3 (Masayuki Ishikawa) contains the full ToMV genome under a T7 promoter. In order to replace the ToMV MP gene with that of ToBRFV, the ToBRFV MP ORF was amplified using primers that overlap the adjacent sequences of ToMV MP (primers 161, 162, Table 1). The ToMV regions between the internal KpnI site and the MP and between the MP and the internal XmaI site were amplified with primers that overlap the ToBRFV MP (primers 158, 160 and 163, 159, respectively, Table 1). The three PCR products then served as templates for fusion PCR (primers 158, 159). The product was digested with KpnI and XmaI and ligated into a similarly digested pTLW3 vector.

In vitro transcription and infection of tomato plants: 2 μl of ToMV pTLW3 and ToMV (MP-TB) were linearized with the restriction enzyme SmaI and cleaned by Gel extraction kit (Zymo Research, ZR-D4002). In-vitro transcription was done according to instructions of the mMESSAGE mMACHINETM T7 kit (Invitrogen by Thermo Fisher Scientific, AM1344). Transcript was diluted 4-fold and then 5 μl of transcript was used to mechanically inoculate N. benthamiana plants that were dusted with carborundum powder prior to inoculation. Systemic infected N. benthamiana leaves were used as inoculum on 2-week-old tomato plants CV. Moneymaker (LA2706, LA3310, UC Davis stock center). Samples were collected 3 weeks after infection.

Construction of TMV-GFP^{MP-ToBRFV}: The binary vector pJL24 (TMV-GFP) contains the full TMV genome with the gene for GFP following the MP gene (Lindbow, 2007). Cloning of MP_{ToBRFV} into this vector was done as follows: The MP_{ToBRFV} ORF was amplified by PCR from infected plants (primers 39, 40, Table 1), adding a sequence overlapping with pJL24 vector at the 5′ and reaching the internal Pad site at the 3′. A second PCR amplified the sequence between the original MP of the TMV-GFP to the internal AgeI site that lies about 1,660 bases upstream of it, adding a sequence overlapping MP_{ToBRFV} (primers 37, 38, Table 1). A 1:1 molar ratio of the two PCR products served as template for fusion PCR (primers 37, 40, Table 1), resulting in a 2,500 bp amplicon, flanked by restriction sites of AgeI and PacI, that was cloned in place of the original sequence. Colonies were then tested for MP_{ToBRFV} using a specific primer (50) and DNA was sequenced with a primer that lies upstream of the MP (41).

### Results

MP_{ToBRFV} is the Tm-2² Resistance-Breaking Factor

To test whether MP_{ToBRFV} is the resistance-breaking factor, the MP sequence of a ToMV infectious clone (Hamamoto et al., 1997) was replaced by MP_{ToBRFV} (ToMV_{MP-ToBRFV} ) (FIG. 1A), In ToMV-sensitive tomato plants (tm-2/tm-2) (FIG. 1B), both ToMV and ToMV_{MP-ToBRFV} were infectious and triggered systemic diseases (FIGS. 1C, D and H). As expected, Tm-2² homozygous tomato plants (FIG. 1E) were immune to ToMV (FIG. 1F and H). However, when these plants were inoculated with ToMV_{MP-ToBRFV}, systemic disease was evident and viral RNA was detected in systemic leaves (FIG. 1G and H). This result established that MP_{ToBRFV} is sufficient to overcome Tm-2² resistance in tomato.

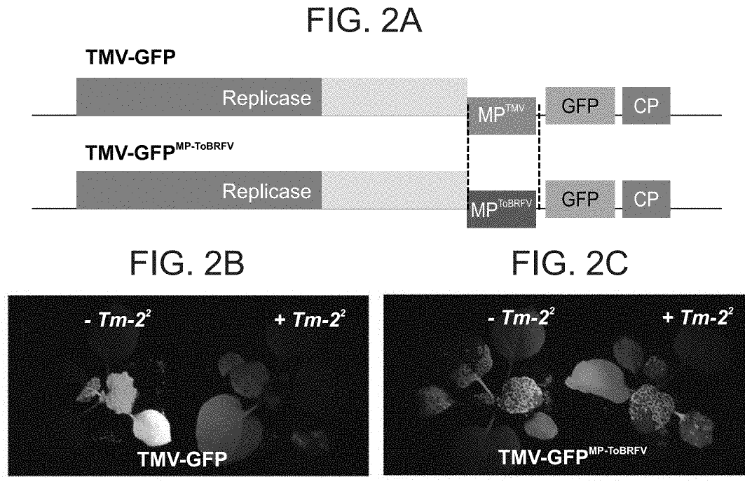

To corroborate that MP_{ToBRFV} is the Tm-2² resistance-breaking factor, a parallel experiment was performed in N. benthamiana. Here, the original MP of the TMV-GFP vector (Lindbow, 2007) was replaced with MP_{ToBRFV} (TMV-GFP_{MP-ToBRFV} ) (FIG. 2A). These viruses were either expressed alone or co-expressed with Tm-2² under the 35S promoter (p35S:Tm-2²) in Nicotiana benthamiana leaves. As expected, Tm-2² conferred resistance against TMV-GFP (FIG. 2B). In marked contrast, Tm-2² did not confer resistance against TMV-GFP_{MP-ToBRFV} (FIG. 2C). Together, these experiments establish that the cause for ToBRFV overcoming Tm-2² resistance is the lack of MP_{ToBRFV} recognition.

MP_{ToBRFV} does not Trigger Tm-2²-Mediated Hypersensitive Response (HR)

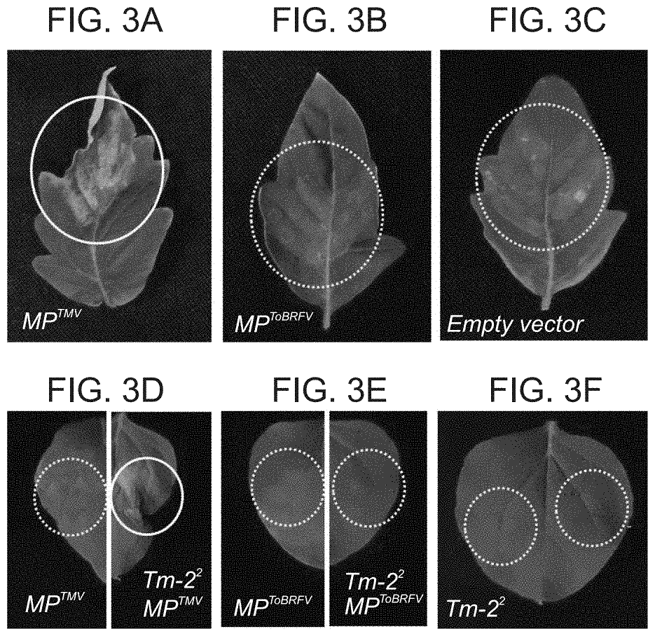

Recognition between Tm-2² and the tobarnovirus MP is a distinct process, which depends on specific elements within both proteins. The present inventors contemplated that specific mutations in Tm-2² may confer the ability to recognize MP_{ToBRFV}. The identification of such modifications requires a robust screening system to identify the activation of Tm-2² immune response. Previously, Farnham and Baulcombe (2006) utilized the appearance of HR necrotic lesions as a hallmark for pathogen recognition and immune response. Since Tm-2² can trigger HR in the presence of MP^{TMV} (Zhang et al,, 2013; Chen et al., 2017; Wang et al., 2020), the present inventors sought to ascertain whether expression of MP_{ToBRFV} will not trigger cell death when co-expressed with Tm-2². To test this hypothesis, the coding sequences of MP_{ToBRFV} and MP^{TMV} were cloned under the control of the 35S promoter 35S: MP_{ToBRFV} and p35S:^{TMV}, respectively) Clones were than transiently expressed in leaflets of tomato cv. Ikram (Tm-2²/tm-2) using agroinfiltration. (FIGS. 3A-C). Cell death was observed in leaflets infiltrated with p35S:MP^{TMV} (FIG. 3A). In contrast, leaflets infiltrated with p35S:MP_{ToBRFV} showed only marginal necrosis (FIG. 3B), similar to the control treatment (FIG. 3C). To generate an efficient screening system for Tm-2² variants, the same system was confirmed in N. benthamiana. Here again, transient co-expression of p35S:MP^{TMV} with p35S:Tm-2² caused tissue necrosis consistent with the establishment of HR (FIG. 3D), while co-expression of p35S:MP_{ToBRFV} with p35S:Tm-2² resulted in no cell death (FIG. 3E), similar to the negative control (FIG. 3F). These results established that the appearance of HR necrosis can be used as a screening platform for the identification of new variants of Tm-2² with MP_{ToBRFV} recognition.

Construction of a Tm-2² Mutant Library using Golden Gate cloning

Next, a Tm-2² mutant library was generated using the golden gate cloning method (Weber et al, 2011), which allows the modular cloning of various components (FIG. 4). Random mutagenesis on the LRR region of Tm-2² was done using error-prone PCR. The mutated LRR parts were then assembled with the CC-NB part of Tm-2² into a level 0 plasmid. The resulting clones were then pooled together and sub-cloned onto a level 2 plant expression cassette which includes the 35S promoter and OCS terminator, resulting in multiple expression clones. These expression clones were then pooled again and transformed to Agrobacterium tuniefaciens to create the Tm-2² mutant library. Next, each of the Agrobacterium isolates was co-infiltrated with p35S:MP_{ToBRFV}. Appearance of necrotic lesions indicated successful recognition of MP_{ToBRFV} and activation of Tm-2²-mediated HR.

Isolation of Tm-2² Mutants that Recognize MP_{ToBRFV}

Out of a total of 1000 screened colonies, 13 Tm-2² mutant alleles, which triggered HR in response MP_{ToBRFV} were isolated and sequenced (FIG. 5A). Interestingly, some of these clones did riot generate HR in response to MP^{TMV}, suggesting a trade-off between the recognition of MP^{TMV} and MP_{ToBRFV} (FIG. 5A). The HR intensity varied between the different clones (FIG. 5B), ranging between mild, moderate and severe. The average mutation rate in the 8 isolated clones was 2.4 mutations per clone, with three clones containing one mutation (colonies 22, 67 and 905), four clones containing 2 mutations (colonies 14, 24, 51, 58 and 67), two clones with 3 mutations (colonies 18 and 547), one clone containing four mutations (colony 872) and one clone containing 5 mutations (colony 184) (FIG. 5B). In addition, six contained only non-synonymous mutation (colonies 14, 22 58, 67, 547 and 905) and seven contained a combination of synonymous and non-synonymous mutations (colonies 18, 24, 51, 65, 184, 311 and 872). Strikingly, three non-synonymous mutations independently repeated in two colonies or more (FIG. 3C). These mutations were in amino-acids F528, S604 and I652. Recurrence of these mutations in more than one colony indicated that they were likely to take part in MP_{ToBRFV} recognition.

To test the function of mutations in F528, S604 and I652, three Tm-2² mutant clones were generated with site-specific mutagenesis in these amino acids: Tm-2² F528S, Tm-2² S604N and Tm-2² I652M (FIGS. 6A-D). Solitary expression of the mutant clones did not have any effect on the leaf. However, their co-expression with MP_{ToBRFV} resulted in the appearance of necrotic lesions (FIGS. 6A-C). These results were similar to expression of the non-mutant Tm-2² gene with MP^{TMV} (FIG. 6D). It is important to note that expression of non-mutant Tm-2² with MP_{ToBRFV} had no effect on the leaf (FIG. 6D). These results suggest that the Tm-2² mutations F528S, S604N and I652M can confer MP_{ToBRFV} recognition and may be used to protect the plant against ToBRFV.

Expression of Tm-2² Mutants Confers Protection against a Resistance-Breaking Virus

The next experiment was aimed to determine if the mutant Tm-2² variants are able to protect against an infection resistance-breaking virus. For that, the different Tm-2² variants were co-expressed with the resistance-breaking TMV-GFP_{MP-ToBRFV} and the progression of viral infection was monitored by GFP fluorescence. Expression of TMV-GFP_{MP-ToBRFV} alone (FIG. 7A), or with non-mutant Tm-2² (FIG. 713), resulted in systemic infection and viral symptoms in young leaves. In marked contrast, co expression of IMV-GFP_{MP-ToBRFV} with the mutated Tm-2² variants resulted in necrosis of the infected leaf, inhibition of systemic infection and lack of symptoms (FIGS. 7C-E). Quantification of GFP fluorescence in the systemic leaves demonstrated that the Tm-2² mutants were able to significantly inhibit the viral infection (FIG. 7F). These results show that expression of the Tm-2² variants can stop the systemic spread of a resistance-breaking tobamovirus, and therefore provide a potential source for ToBRFV resistance.

### Structural Changes in T-2² Conferred by the Different Mutations

The enhancement of Tm-2² function is likely caused by modifications of protein structure, which enable Tm-2² to better bind or relay the immune signal in response to MP_{ToBRFV}. To explore the structural changes caused by the Tm-2² mutations, 3D homology modelling was performed. based on the recently published structure of the Arabidopsis NLR ZAR1 (Wang et al., 2019) (FIG. 8). According to this model, the spatial location of all three mutationsis on the convex side of the LRR domain, suggesting that changes in this region can determine MP recognition specificity. F528 and S604 are both located in a-helixes, and the S604M mutation leads to the disruption of this structure. I652 is located in one of two loop structures extending from the convex side of the NLR. All three mutations lead to changes in the positions of these loop structures, which possibly play a central role in MP recognition.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

Broadbent L (1976). Epidemiology and control of tomato mosaic virus. Annu Rev Phytopathol, 14: 75-96.

Cambrón-Crisantos, J. M., Rodríguez-Mendoza, J., Valencia-Luna, J. B., Rangel, S. A., de Jesús García-Ávila, C., & López-Buenfil, J. A. (2018). First report of Tomato brown rugose fruit virus (ToBRFV) in Michoacan, Mexico. Mexican Journal of Phytopathology, 37(1), 185-192.

Chen, T., Liu, D., Niu, X., Wang, J., Qian, L., Han, L., . . . & Liu, Y. (2017). Antiviral resistance protein Tm-22 functions on the plasma membrane. Plant physiology, 173(4), 2399-2410.

Dahan-Meir, T., Filler-Hayut, S., Melamed-Bessudo, C., Bocobza, S., Czosnek, H., Aharoni, A., & Levy, A. A. (2018). Efficient in planta gene targeting in tomato using geminiviral replicons and the CRISPR/Cas9 system. The Plant Journal, 95(1), 5-16.

Farnham G, Baulcombe DC (2006). Artificial evolution extends the spectrum of viruses that are targeted by a disease-resistance gene from potato. Proc Natl Acad Sci USA, 103: 18828-18833.

Fidan, H., Sarikaya, P., & Calis, O. (2019). First report of Tomato brown rugose fruit virus on tomato in Turkey. New Disease Reports, 39(18), 2044-0588.

Hamamoto, H., Watanabe, Y., Kamada, H., & Okada, Y. (1997). Amino acid changes in the putative replicase of tomato mosaic Tobamovirus that overcome resistance in Tm-1 tomato. Journal of general virology, 78(2), 461-464.

Harris, C. J., Slootweg, E. J., Goverse, A., & Baulcombe, D. C. (2013). Stepwise artificial evolution of a plant disease resistance gene. Proceedings of the National Academy of Sciences, 110(52), 21189-21194.

Ishibashi, K., Masuda, K., Naito, S., Meshi, T., & Ishikawa, M. (2007). An inhibitor of viral RNA replication is encoded by a plant resistance gene. Proceedings of the National Academy of Sciences, 104(34), 13833-13838.

Kobayashi, M., Yamamoto-Katou, A., Katou, S., Hirai, K., Meshi, T., Ohashi, Y., & Mitsuhara, I. (2011). Identification of an amino acid residue required for differential recognition of a viral movement protein by the Tomato mosaic virus resistance gene Tm-22. Journal of plant physiology, 168(10), 1142-1145.

Lanfermeijer, F. C., Dijkhuis, J., Sturre, M. J., de Haan, P., & Hille, J. (2003). Cloning and characterization of the durable tomato mosaic virus resistance gene Tm-2 2 from Lycopersicon esculentum. Plant molecular biology, 52(5), 1039-1051.

Lanfermeijer, F. C., Warmink, J., & Hille, J. (2005). The products of the broken Tm-2 and the durable Tm-2 2 resistance genes from tomato differ in four amino acids. Journal of experimental botany, 56(421), 2925-2933.

Lindbo, J. A. (2007). High-efficiency protein expression in plants from agroinfection-compatible Tobacco mosaic virusexpression vectors. BMC biotechnology, 7(1), 52.

Ling, K. S., Tian, T., Gurung, S., Salati, R., & Gilliard, A. (2019). First report of tomato brown rugose fruit virus infecting greenhouse tomato in the United States. Plant Disease, 103(6), 1439.

Luria, N., Smith, E., Reingold, V., Bekelman, I., Lapidot, M., Levin, I., . . . & Dombrovsky, A (2017). A new Israeli Tobamovirus isolate infects tomato plants harboring Tm-2² resistance genes. PloS one, 12(1), e0170429.

Maayan, Y., Pandaranayaka, E. P., Srivastava, D. A., Lapidot, M., Levin, I., Dombrovsky, A., & Harel, A. (2018). Using genomic analysis to identify tomato Tm-2 resistance-breaking mutations and their underlying evolutionary path in a new and emerging Tobamovirus. Archives of virology, 163(7), 1863-1875.

Menzel, W., Knierim, D., Winter, S., Hamacher, J., & Heupel, M. (2019). First report of tomato brown rugose fruit virus infecting tomato in Germany. New Disease Reports, 39(1), 2044-0588.

Meshi, T., Motoyoshi, F., Maeda, T., Yoshiwoka, S., Watanabe, H., & Okada, Y. (1989). Mutations in the tobacco mosaic virus 30-kD protein gene overcome Tm-2 resistance in tomato. The Plant Cell, 1(5), 515-522.

Padmanabhan, M. S., & Dinesh-Kumar, S. P. (2014). The conformational and subcellular compartmental dance of plant NLRs during viral recognition and defense signaling. Current opinion in microbiology, 20, 55-61.

Panno, S., Caruso, A. G., & Davino, S. (2019). First report of tomato brown rugose fruit virus on tomato crops in Italy. Plant Disease, 103(6), 1443-1443.

Romero PA, Arnold FH. (2009). Exploring protein fitness landscapes by directed evolution. Nat Rev Mol Cell Biol, 10(12), 866.

Salem, N., Mansour, A., Ciuffo, M., Falk, B. W., & Turina, M. (2016). A new tobamovirus infecting tomato crops in Jordan. Archives of virology, 161(2), 503-506.

Segretin, M. E., Pais, M., Franceschetti, M., Chaparro-Garcia, A., Bos, J. I., Banfield, M. J., & Kamoun, S. (2014). Single amino acid mutations in the potato immune receptor R3a expand response to Phytophthora effectors. Molecular Plant-Microbe Interactions, 27(7), 624-637.

Skelton, A., Buxton-Kirk, A., Ward, R., Harju, V., Frew, L., Fowkes, A., . . . & Pufal, H. (2019). First report of Tomato brown rugose fruit virus in tomato in the United Kingdom. New Disease Reports, 40, 12-12.

Strasser, M., & Pfitzner, A. J. P. (2007). The double-resistance-breaking Tomato mosaic virus strain ToMV1-2 contains two independent single resistance-breaking domains. Archives of virology, 152(5), 903.

Wang, J., Chen, T., Han, M., Qian, L., Li, J., Wu, M., . . . & Hong, Y. (2020). Plant NLR immune receptor Tm-2² activation requires NB-ARC domain-mediated self-association of CC domain. PLoS pathogens, 16(4), e1008475.

Weber, E., Engler, C., Gruetzner, R., Werner, S., & Marillonnet, S. (2011). A modular cloning system for standardized assembly of multigene constructs. PloS one, 6(2).

Weber, H., Ohnesorge, S., Silber, M. V., & Pfitzner, A. J. P. (2004). The Tomato mosaic virus 30 kDa movement protein interacts differentially with the resistance genes Tm-2 and Tm-2 2. Archives of virology, 149(8), 1499-1514.

Weber, H. A. N. S., Schultze, S. A. B. I. N. E., & Pfitzner, A. J. (1993). Two amino acid substitutions in the tomato mosaic virus 30-kilodalton movement protein confer the ability to overcome the Tm-2 (2) resistance gene in the tomato. Journal of virology, 67(11), 6432-6438.

Xu, H., Petersen, E. I., Petersen, S. B., & El-Gewely, M. R. (1999). Random mutagenesis libraries: optimization and simplification by PCR. Biotechniques, 27(6), 1102-1108.

Yan, Z. Y., Ma, H. Y., Han, S. L., Geng, C., Tian, Y. P., & Li, X. D. (2019). First report of tomato brown rugose fruit virus infecting tomato in China. Plant Disease, 103(11), 2973-2973.

Zhang, H., Zhao, J., Liu, S., Zhang, D. P., & Liu, Y. (2013). Tm-2² confers different resistance responses against tobacco mosaic virus dependent on its expression level. Molecular plant, 6(3), 971-974.

What is claimed is:

1. A tomato plant or a part thereof, expressing a Tm-2² protein having an amino acid sequence which renders the plant resistant to tomato brown rugose fruit virus (ToBRFV).

2. The tomato plant of claim 1, being homozygotic for a Tm-2² mutation, which renders the plant resistant to tomato brown rugose fruit virus (ToBRFV).

3. The tomato plant of claim 2, wherein said mutation comprises an amino acid modification which enhances immune activation thereof by the movement protein (MP) of said ToBRFV as compared to the wild-type Tm-2² protein.

4. The tomato plant of claim 1, wherein said Tm-2² protein comprises an amino acid modification which enhances immune activation thereof by the movement protein (MP) of said ToBRFV as compared to the wild-type Tm-2² protein.

5. The tomato plant of claim 4, wherein said amino acid modification is in the leucine rich repeat (LRR) domain of said Tm-2² protein.

6. The tomato plant of claim 5, wherein the Tm-2² protein comprises an amino acid modification at any one of positions 528, 604 or 652, as compared to the wild-type Tm-2² protein.

7. The tomato plant of claim 6, wherein said modification is a substitution.

8. The tomato plant of claim 7, wherein said modification at position 528 is F5285.

9. The tomato plant of claim 7, wherein said modification at position 604 is 5604N.

10. The tomato plant of claim 7, wherein said modification at position 652 is I652M.

11. The tomato plant of claim 1, being resistant to tomato mosaic virus (ToMV) and tobacco mosaic virus (TMV).

12. A cutting of a tomato plant of claim 1.

13. A seed of the plant of claim 2.

14. A cell having the genome of the plant of claim 2.

15. A method of breeding a tomato plant, comprising crossing the plant of claim 2 with an additional tomato plant, thereby breeding the tomato plant.

16. The method of claim 15, wherein said crossing comprising pollinating.

17. A hybrid seed produced by the method of claim 15.

18. A hybrid plant, or part thereof, produced by growing the hybrid seed of claim 17.

19. A method of growing a plant, comprising vegetatively propagating the plant of claim 1, thereby growing the plant.

20. A food of processed product comprising the plant of claim 1 or parts thereof.
